(19) United States
(12) Patent Application Publication
(10) Pub. No.: US 2023/0402131 A1
(43) Pub. Date: Dec. 14, 2023

(54) BIOMARKER AND DIAGNOSIS SYSTEM FOR COLORECTAL CANCER DETECTION

(71) Applicant: Hangzhou Calibra Diagnostics Co., Ltd., Hangzhou (CN)

(72) Inventors: Rongchang CHEN, Hangzhou (CN); Sheng QUAN, Hangzhou (CN); Chao ZHANG, Hangzhou (CN); Ziqing KONG, Hangzhou (CN); Pengyun LIU, Hangzhou (CN); Huafen LIU, Hangzhou (CN)

(21) Appl. No.: 18/073,834

(22) Filed: Dec. 2, 2022

(30) Foreign Application Priority Data
Jun. 10, 2022 (CN) 202210658811.5
Jun. 10, 2022 (CN) 202210661330.X

(51) Int. Cl. G16B 40/00; G01N 33/68; G01N 33/53; G16H 50/30; G16B 5/20; G16C 20/70

(52) U.S. Cl. CPC G16B 40/00; G01N 33/6848; G01N 33/5308; G16H 50/30; G16B 5/20; G16C 20/70

(57) ABSTRACT

The present disclosure provides a biomarker for detecting colorectal cancer and a use thereof. A metabolomics method is used to analyze metabolites with significant differences in urine of patients with colorectal cancer and normal people, such that a series of biomarkers capable of early predicting an occurrence risk of colorectal cancer are screened out, a group of biomarkers are further screened to construct a diagnostic model for colorectal cancer, and the model can be used for conveniently, non-invasively and effectively predicting whether an individual suffers from colorectal cancer, and meets clinical needs.

### CROSS-REFERENCE TO RELATED APPLICATIONS

This patent application claims the priority of Chinese Patent Application No. 202210661330.X, filed on Jun. 10, 2022 and Chinese Patent Application No. 202210658811.5, filed on Jun. 10, 2022, the description, claims, abstract, and drawings of which are applied as part of the present disclosure.

### TECHNICAL FIELD

The present disclosure relates to the field of medicine and use of metabolomics to screen biomarkers for colorectal cancer and use of the biomarkers in diagnosing colorectal cancer, in particular to a biomarker capable of predicting the risk of colorectal cancer by detecting a urine sample.

### BACKGROUND OF THE INVENTION

Metabolomics is a subject for qualitatively and quantitatively analyzing small molecule metabolites with a relative molecular weight less than 1,000 in the body or body fluid. The physiological and pathological conditions of the body can be reflected through a metabolomics analysis, and differences among different individuals can also be distinguished. With the development of mass spectrometry technology, liquid chromatography-mass spectrometry (LC-MS) has become the most important research tool in metabolomics research. At present, metabolomics has been widely used in the field of clinical diagnosis, mainly for discovering metabolic markers related to disease diagnosis and treatment.

Colorectal cancer (CRC) is one of the most common malignancies in China and worldwide. The Cancer statistics in China, 2018 shows that the morbidity and mortality of colorectal cancer in China respectively rank the 3rd and 5th in all malignant tumors, wherein there are 376 thousands of new cases and 191 thousands of death cases. According to the “Chinese experts consensus on early diagnosis and early treatment of colorectal cancer” in 2020, the incidence of colorectal cancer in China leaps 2nd (33.17/100 thousand) of that of malignant tumors in cities and the mortality 4th (15.98/100 thousand). The incidence (19.71/100 thousand) and mortality (9.68/100 thousand) of malignant tumors in rural areas rank 5th. The incidence of colorectal cancer is increasing year by year in almost all tumor-registered areas of the country. Although prevention and treatment of colorectal cancer has advanced to some extent through long-term basic research and clinical practice, the overall five-year survival rate remains low. The reasons include the lack of effective biomarkers for early predict of the risk of CRC development. Therefore, early discovery and early treatment are also the key to improving the overall survival of colorectal cancer.

At present, the diagnosis of colorectal cancer is mainly performed by enteroscopy and imaging. In the course of research and discovery of cancer biomarkers, various Omics technologies based on system biology also play an important role. Biomarkers found by the results of genomics and proteomics research have been applied to cancer research. For example, an in-vitro gene diagnosis kit for detecting KRAS gene mutation and BMP3/NDRG4 gene methylation of colorectal cancer, namely, “KRAS gene mutation and BMP3/NDRG4 gene methylation and fecal occult blood combined detection kit (PCR fluorescent probe-colloidal gold method)” has been approved by the National Medical Products Administration for marketing on Nov. 9, 2020, and is used in screening high-risk populations with poor compliance of colorectal cancer.

A large number of research results from metabolomics research in recent years are being found more and more widely in various academic journals. In 2014, Cross et al. performed a metabolomics study of serums of 254 patients with colorectal cancer and matched 254 disease-free controls. No specific metabolites directly related to rectal cancer risk were screened from 447 identified serum metabolites. However, interestingly, it was found that the glycochenodeoxycholate content in bile acid was significantly positively correlated with the risk of rectal cancer in the female population. In another metabolomics study for colorectal cancer, Long et al. first performed a non-targeted metabolomics study of the serums of 30 patients with CRC and 30 healthy controls. The few studies on early discovery and early warning of CRC above theoretically demonstrated the feasibility of finding CRC-related metabolic biomarkers through metabolomics techniques. However, a blood sample is required by the metabolic biomarkers for colorectal cancer reported at present, while a fecal sample is required for gene detection for a colorectal cancer risk. Both sample types have no advantages in noninvasive and simple sample collection.

Therefore, it is urgent to find a biomarker capable of performing noninvasive sampling conveniently and rapidly and early prediction whether an individual has a risk of colorectal cancer, thereby more efficiently evaluating the risk of colorectal cancer.

### SUMMARY OF THE INVENTION

Aiming at the problems existing in the prior art, the present disclosure provides a biomarker for detecting colorectal cancer. A metabolomics method is used to analyze metabolites with significant differences in urine of patients with colorectal cancer and normal people, such that a series of biomarkers capable of early predicting an occurrence risk of colorectal cancer (CRC) are screened out, a group of biomarkers are further screened to construct a diagnostic model for colorectal cancer, and the model can be used for conveniently, non-invasively and effectively predicting whether an individual suffers from colorectal cancer, and meets clinical needs.

In one aspect, the present disclosure provides a method for testing whether an individual suffers from colorectal cancer comprising test a biomarker in the liquid sample as to determine the amounts of the biomarker, wherein the biomarker is selected from one or more of the following: 2-piperidinone, 3-hydroxyanthranilate, 3-indoxyl sulfate, 4-hydroxyphenylacetylglutamine, 4-hydroxyphenylpyruvate, 5-hydroxyindole glucuronide, 6-hydroxyindole sulfate, dimethylguanidinovaleric acid, N-acetyl-cadaverine, N-formylmethionine, nicotinamide, nicotinamide N-oxide, N-methyl-4-aminobutyric acid, p-cresol glucuronide, p-cresol sulfate, phenylacetylalanine, phenylacetylglutamate, phenylacetylglutamine, phenylacetylhistidine, phenylacetylmethionine, phenylacetylserine, phenylacetyltaurine, phenylacetylthreonine, trimethylamine N-oxide, xanthine, and trizma acetate.

Through a non-targeted metabolomics research, urine samples of a healthy group and a colorectal cancer patient group are analyzed by using an ultra-performance liquid chromatography-tandem mass spectrometry (UPLC-MS/MS). Metabolites with significant differences between a colorectal cancer sample and a control sample are respectively screened by using four statistical methods of random forest, PLS-DA, difference test and SVM. The screened metabolites with significant differences in the four statistical analysis methods are selected, and finally 26 urine metabolites are obtained and used as biomarkers for efficiently predicting whether an individual suffers from colorectal cancer.

In some embodiments, the biomarker for predicting whether an individual suffers from colorectal cancer may be a detection target to prepare a detection reagent, such as a sample pretreatment reagent, an antigen or an antibody, and other biological reagent and kit suitable for detecting the biomarker; and a standardized reagent or a kit and the like can also be developed to be suitable for the detection of the biomarker by LC-UV or LC-MS.

In some embodiments, the biomarker of the present disclosure is obtained by screening urine samples, and thus is particularly suitable for being developed into a urine detection reagent or kit for predicting colorectal cancer, and the like.

In some embodiments, when the selected biomarker is an amino acid or an amino acid derivative or contains an amino group, such as 4-hydroxyphenylacetylglutamine, N-acetyl-cadaverine, N-formylmethionine, N-methyl-4-aminobutyric acid, phenylacetylalanine, phenylacetylglutamate, phenylacetylhistidine, phenylacetylmethionine, phenylacetylserine, phenylacetyltaurine, and phenylacetylthreonine. A PITC method, or an AQC method, or an OPA method, or an FMOC method and other amino acid analysis method can be combined to prepare a reagent or a kit for detecting these biomarkers suitable for use in an amino acid analyzer or by LC-UV.

Furthermore, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, phenylacetylthreonine, 3-hydroxyanthranilate, 5-hydroxyindole glucuronide, phenylacetylglutamate, phenylacetylhistidine, 2-piperidinone, N-formylmethionine, phenylacetyltaurine, 3-indoxyl sulfate, 6-hydroxyindole sulfate, and trimethylamine N-oxide.

By examining the concentration changes of the biomarkers in the urine of patients with colorectal cancer and normal people, and performing sorting according to the fold changes, 20 biomarkers with the largest fold change between the patients with colorectal cancer and normal people (theoretically, the compounds with the largest fold change can be the most effective markers) are further selected from 26 biomarkers, and can be used for more effectively distinguishing or predicting the risk of colorectal cancer or constructing a diagnostic model of colorectal cancer.

Furthermore, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, and phenylacetylthreonine.

By examining the concentration changes of the biomarkers in the urine of patients with colorectal cancer and normal people, and performing sorting according to the fold changes, 10 biomarkers with the largest fold change between the patients with colorectal cancer and normal people (theoretically, the compounds with the largest fold change may possible be the most effective markers) are further selected from 26 biomarkers, and can be used for more effectively distinguishing or predicting the risk of colorectal cancer or constructing a diagnostic model of colorectal cancer.

Furthermore, the biomarker is selected from one or more of the following: 4-hydroxyphenylpyruvate, N-methyl-4-aminobutyric acid, p-cresol sulfate, phenylacetylmethionine, and phenylacetylthreonine.

By examining the concentration changes of the biomarkers in the urine of patients with colorectal cancer and normal people, and performing sorting according to the fold changes, 5 biomarkers with the largest fold change between the patients with colorectal cancer and normal people (theoretically, the compounds with the largest fold change may possible be the most effective markers) are further selected from 26 biomarkers, and can be used for more effectively distinguishing or predicting the risk of colorectal cancer or constructing a diagnostic model of colorectal cancer.

Furthermore, the biomarker is selected from one or more of the following: p-cresol sulfate and phenylacetylthreonine.

By examining the concentration changes of the biomarkers in the urine of patients with colorectal cancer and normal people, and performing sorting according to the fold changes, 2 biomarkers with the largest fold change between the patients with colorectal cancer and normal people (theoretically, the compounds with the largest fold change may possible be the most effective markers) are further selected from 26 biomarkers, and can be used for more effectively distinguishing or predicting the risk of colorectal cancer or constructing a diagnostic model of colorectal cancer.

Furthermore, the reagent is used for detecting biomarkers in urine.

In the present disclosure, biomarkers for colorectal cancer are screened from urine and have significant difference in the urine of patients with colon cancer and patients without colon cancer. By collecting urine samples, the biomarkers in the urine of an individual can be detected to predict or assist to diagnose whether the individual suffers from colorectal cancer or the possibility of the individual suffering from colorectal cancer, or the biomarkers in the urine of a certain group can be detected so as to classify the group into a colorectal cancer group or a non-colorectal cancer group. Compared with blood and feces, urine is non-invasive and simple to collect. Using the urine biomarkers in the preparation of a diagnostic reagent for colorectal cancer or in the diagnosis of colorectal cancer will have greater advantages and prospects.

Furthermore, the detection of the biomarker in urine is to detect the presence or relative abundance or concentration of the biomarker in the urine sample of the individual.

In some embodiments, the relative abundance is preferably used and is the peak area of the biomarker in a detection spectrum obtained by an ultra-performance liquid chromatography-tandem mass spectrometry. For example, if the average peak area of a biomarker in a control sample (an individual not suffering from colon cancer) is 500 and the average peak area in a colon cancer sample is 3,000, the abundance of the biomarker in the colon cancer sample is considered to be 6 times that in the control sample.

In the second aspect, the present disclosure provides a kit or a chip for predicting whether an individual suffers from colorectal cancer. The kit or chip comprises a detection reagent of the above biomarkers.

Furthermore, the reagent is used for detecting biomarkers in urine.

In the third aspect, the present disclosure provides a biomarker combination for predicting whether an individual suffers from colorectal cancer, wherein the biomarker combination comprises the following biomarkers: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, and phenylacetylthreonine.

Furthermore, the biomarker combination comprises the following biomarkers: 2-piperidinone, 3-hydroxyanthranilate, 3-indoxyl sulfate, 4-hydroxyphenylacetylglutamine, 4-hydroxyphenylpyruvate, 5-hydroxyindole glucuronide, 6-hydroxyindole sulfate, dimethylguanidinovaleric acid, N-acetyl-cadaverine, N-formylmethionine, nicotinamide, nicotinamide N-oxide, N-methyl-4-aminobutyric acid, p-cresol glucuronide, p-cresol sulfate, phenylacetylalanine, phenylacetylglutamate, phenylacetylglutamine, phenylacetylhistidine, phenylacetylmethionine, phenylacetylserine, phenylacetyltaurine, phenylacetylthreonine, trimethylamine N-oxide, xanthine, and trizma acetate.

In the fourth aspect, the present disclosure provides a system for predicting whether an individual suffers from colorectal cancer, wherein the system comprises a data analysis module; and the data analysis module is used for analyzing a detection value of a biomarker, and the biomarker is selected from one or more of the following: 2-piperidinone, 3-hydroxyanthranilate, 3-indoxyl sulfate, 4-hydroxyphenylacetylglutamine, 4-hydroxyphenylpyruvate, 5-hydroxyindole glucuronide, 6-hydroxyindole sulfate, dimethylguanidinovaleric acid, N-acetyl-cadaverine, N-formylmethionine, nicotinamide, nicotinamide N-oxide, N-methyl-4-aminobutyric acid, p-cresol glucuronide, p-cresol sulfate, phenylacetylalanine, phenylacetylglutamate, phenylacetylglutamine, phenylacetylhistidine, phenylacetylmethionine, phenylacetylserine, Phenylacetyltaurine, phenylacetylthreonine, trimethylamine N-oxide, xanthine, and trizma acetate.

Furthermore, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, phenylacetylthreonine, 3-hydroxyanthranilate, 5-hydroxyindole glucuronide, phenylacetylglutamate, phenylacetylhistidine, 2-piperidinone, N-formylmethionine, phenylacetyltaurine, 3-indoxyl sulfate, 6-hydroxyindole sulfate, and trimethylamine N-oxide.

Furthermore, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, and phenylacetylthreonine.

Furthermore, a detection value of the biomarker is obtained by detecting the biomarker in urine.

Furthermore, a detection value of the biomarker is obtained by detecting the presence or relative abundance or concentration of the biomarker in the urine sample of the individual.

Furthermore, the data analysis module uses a random forest or a logistic regression equation to construct a model for analysis.

Furthermore, the data analysis module calculates a predictive value for predicting whether an individual suffers from colorectal cancer by substituting a detection value of the biomarker into a logistic regression equation to evaluate whether the individual suffers from colorectal cancer.

Furthermore, the logistic regression equation is as follows:

Z=4-hydroxyphenylpyruvate*0.037986+dimethylguanidinovaleric acid*0.4818−N-methyl-4-aminobutyric acid*1.0077−nicotinamide*1.525−p-cresol glucuronide*0.0353−p-cresol sulfate*0.021798−phenylacetylalanine*0.1902+phenylacetylglutamine*0.858−phenylacetylmethionine*0.118805+phenylacetylthreonine*0.59727+0.7486,

$$p = \frac{1}{1 + {e}^{z}}$$

wherein e is the base of the natural logarithm; and p is a predictive value for predicting whether an individual suffers from colorectal cancer.

e is the base of the natural logarithm and an infinite non-repeating decimal, has a value of 2.71828 . . . , and is defined as when n->∞, a limit of

$$\\ ( 1 + 1 / n ) ( {lim}_{n -> ∞} {( 1 + \frac{1}{n} )}^{n} ) .$$

The name of the biomarker represents the relative abundance of the corresponding biomarker in a urine sample, that is, a peak area of the biomarker in a detection spectrum obtained by an ultra-performance liquid chromatography-tandem mass spectrometry.

Furthermore, when p is greater than 0.5, the individual is predicted to have a high probability of colorectal cancer; and when p is less than 0.5, the individual is predicted to have a low probability of colorectal cancer.

In the further aspect, the present disclosure provides use of the above system in constructing a detection model of a probability value for predicting whether an individual suffers from colorectal cancer.

In another aspect, the present disclosure provides a method for diagnosing or predicting whether an individual suffers from colorectal cancer. The method comprises: providing a biological sample for an individual; detecting whether a following biomarker existing in the sample, wherein the biomarker is selected from one or more of the following: 2-piperidinone, 3-hydroxyanthranilate, 3-indoxyl sulfate, 4-hydroxyphenylacetylglutamine, 4-hydroxyphenylpyruvate, 5-hydroxyindole glucuronide, 6-hydroxyindole sulfate, dimethylguanidinovaleric acid, N-acetyl-cadaverine, N-formylmethionine, nicotinamide, nicotinamide N-oxide, N-methyl-4-aminobutyric acid, p-cresol glucuronide, p-cresol sulfate, phenylacetylalanine, phenylacetylglutamate, phenylacetylglutamine, phenylacetylhistidine, phenylacetylmethionine, phenylacetylserine, phenylacetyltaurine, phenylacetylthreonine, trimethylamine N-oxide, xanthine, and trizma acetate; when the content of the biomarker in the blood sample exceeds a threshold, it indicates that the individual suffers from colorectal cancer or has a high risk of suffering from colorectal cancer; and when the content of the biomarker in the blood sample is lower than the threshold, it indicates that the individual does not suffer from colorectal cancer or has a low risk of suffering from colorectal cancer.

In some embodiments, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, phenylacetylthreonine, 3-hydroxyanthranilate, 5-hydroxyindole glucuronide, phenylacetylglutamate, phenylacetylhistidine, 2-piperidinone, N-formylmethionine, phenylacetyltaurine, 3-indoxyl sulfate, 6-hydroxyindole sulfate, and trimethylamine N-oxide.

In some embodiments, the biomarker is selected from one or more of the following: 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, and phenylacetylthreonine.

In some embodiments, the biomarker is selected from one or more of the following: 4-hydroxyphenylpyruvate, N-methyl-4-aminobutyric acid, p-cresol sulfate, phenylacetylmethionine, and phenylacetylthreonine.

In some embodiments, the biomarker is selected from one or more of the following: p-cresol sulfate and phenylacetylthreonine.

In some embodiments, the sample is a urine sample. In some embodiments, the detection method comprises analysis by an ultra-performance liquid chromatography-tandem mass spectrometry (UPLC-MS/MS). In some embodiments, the detection of the biomarker in urine is to detect the presence or relative abundance or concentration of the biomarker in the urine sample of the individual.

The present disclosure has the following beneficial effects:
1. 26 whole new biomarkers capable of predicting the occurrence risk of colorectal cancer (CRC) at an early stage are screened;
2. 2, 3, 5, 10, 20, and 26 biomarkers are screened to construct a random forest diagnosis model for colorectal cancer and it is found that 10 biomarkers are the best for constructing a model for colorectal cancer;
3. By comparing the random forest model and the logistic regression model constructed with 10 biomarkers, it is found that the logistic regression model could further improve the detection accuracy and could be used to more effectively predict whether an individual suffers from colorectal cancer, with the AUC value reaching 0.957; and
4. It is non-invasive and more convenient to detect only by collecting urine samples, and compared with detecting by serum or feces samples, the detection by urine has greater advantages and prospects.

### BRIEF DESCRIPTION OF DRAWINGS

FIG. 1 is the flow chart of screening biomarkers in urine by metabolomics in example 1;

FIG. 2 shows the structural formula of 3-indoxyl sulfate in example 1;

FIG. 3 shows the structural formula of 4-hydroxyphenylacetylglutamine in example 1;

FIG. 4 shows the structural formula of 5-hydroxyindole glucuronide in example 1;

FIG. 5 shows the structural formula of phenylacetylglutamate in example 1;

FIG. 6 shows the structural formula of phenylacetylhistidine in example 1;

FIG. 7 shows the structural formula of phenylacetylmethionine in example 1;

FIG. 8 shows the structural formula of phenylacetylthreonine in example 1;

FIG. 9 is the schematic diagram of comparison of prediction accuracy of a colorectal cancer diagnostic model constructed by selecting 2, 3, 5, 10, 20, and 26 biomarkers respectively from 26 biomarkers in example 2;

FIG. 10 shows an ROC curve of a random forest model for predicting colorectal cancer constructed in example 2;

FIG. 11 is an analysis map of the random forest model for predicting colorectal cancer in example 2;

FIG. 12 an ROC curve of a logistic regression model for predicting colorectal cancer constructed in example 2;

FIG. 13 is an analysis map of the logistic regression model for predicting colorectal cancer in example 2; and

FIG. 14 shows an accuracy evaluation result of a colorectal cancer model in example 3.

### DETAILED DESCRIPTION OF THE INVENTION

The present disclosure is further described in detail below with reference to the accompanying drawings and examples. It should be pointed out that the following examples are intended to facilitate the understanding of the present disclosure without any limitation. The reagents used in the examples are known and commercially available products.

### Example 1 Screening Biomarkers of Colorectal Cancer in Urine by Metabolomics

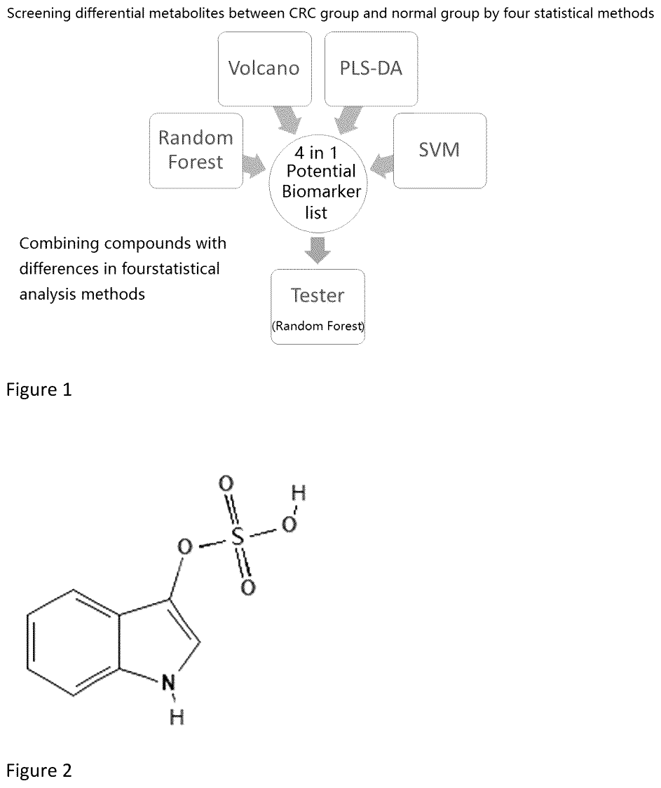

In the example, through a non-targeted metabolomics research, urine samples of a healthy group and a colorectal cancer patient group were analyzed by using an ultra-performance liquid chromatography-tandem mass spectrometry (UPLC-MS/MS). Besides, metabolites with significant differences between a colorectal cancer sample and a control sample were respectively screened by using four statistical methods of random forest, PLS-DA, volcano, and SVM. The screened metabolites with significant differences in the four statistical analysis methods were selected, finally 26 urine metabolites were obtained and used as biomarkers, and the functions of the biomarkers in the diagnosis or distinguishment of colorectal cancer were verified (see FIG. 1 for the flow chart).

Specific steps were as follows:

1. Experimental Method

(1) Sample Collection

Urine samples were collected from 50 patients with colorectal cancer and 50 control individuals (non-colorectal cancer individuals). The patients with colorectal cancer were individuals with colorectal cancer confirmed by a colonoscopy.

(2) Sample Treatment

Methanol was added into the urine samples in a proportion of 1:4, the urine samples were shaken for 3 min to be mixed well, and the mixture was centrifuged at 20° C. and 4,000×g for 10 min. 100 μL of supernatant of each of 4 samples was put into 4 sample plates and blow-dried with nitrogen, and a complex solution was added for a subsequent LC-MS/MS detection.

(3) LC-MS/MS Detection and Data Processing

m/z ions were extracted from original mass spectrometry data detected by LC-MS/MS, a database was searched to retrieve and identify metabolites, chromatographic peak integrals of the metabolites were examined to obtain peak areas, data normalization and missing value filling were performed to obtain a data matrix to perform subsequent bioinformatic analysis, including four statistical methods of random forest, PLS-DA (partial least squares), volcano (volcano plot), and SVM (support vector machine), and the most effective differential metabolite ranking lists for sample grouping were respectively screened between the colorectal cancer samples and the control samples. Finally, the metabolites screened in the four methods were selected as biomarkers for colorectal cancer.

2. Experimental Results

32, 41, 35, and 52 different metabolites were screened by four statistical methods of random forest, PLS-DA, difference test and SVM, wherein 26 metabolites, i.e. 26 biomarkers, were screened in the four data analysis methods, as shown in Table 1.

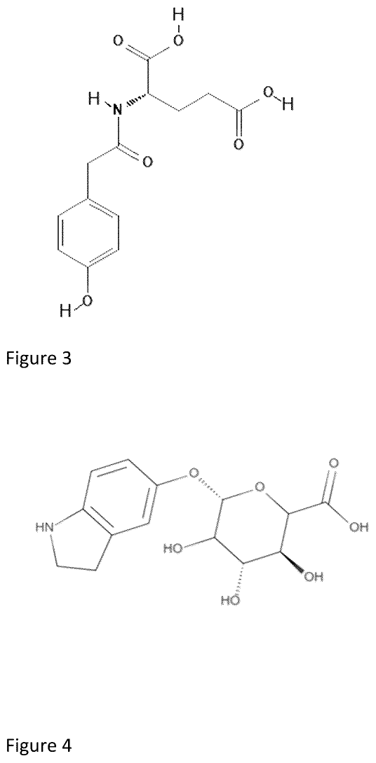

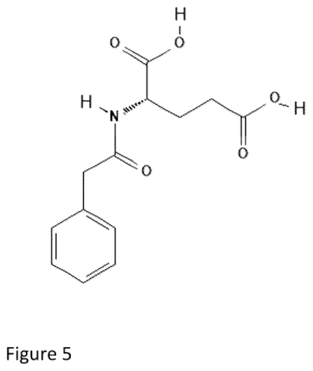

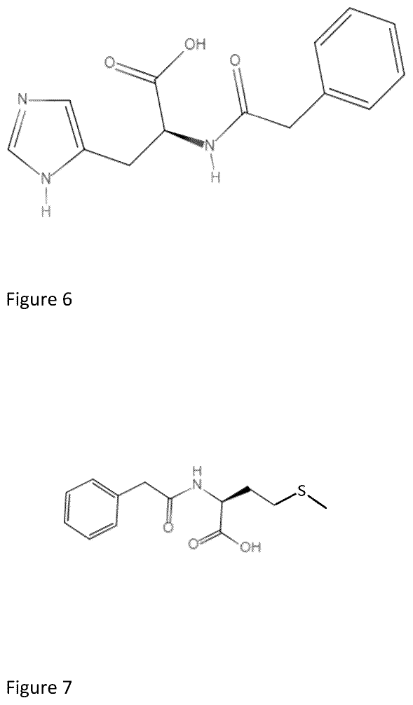

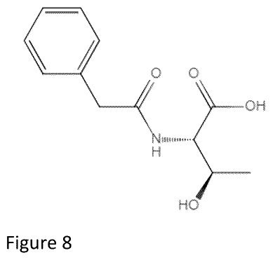

TABLE 1
25 biomarkers for colorectal cancer

| Serial | | | |
|---|---|---|---|
| No. | English Name | CAS code | Molecular formula |
| 1 | 2-piperidinone | 675-20-7 | C₅H₉NO |
| 2 | 3-hydroxyanthranilate | 548-93-6 | C₇H₇NO₃ |
| 3 | 3-indoxyl sulfate | — | C₈H₇NO₄S |
| | | | (structural formula |
| | | | shown in FIG. 2) |
| 4 | 4-hydroxyphenyl- | — | C₁₃H₁₅NO₆ |
| | acetylglutamine | | (structural formula |
| | | | shown in FIG. 3) |
| 5 | 4-hydroxyphenylpyruvate | 156-39-8 | C₉H₈O₄ |
| 6 | 5-hydroxyindole glucuronide | — | C₈H₇NOC₆H₈O₆ |
| | | | (structural formula |
| | | | shown in FIG. 4) |
| 7 | 6-hydroxyindole sulfate | 487-94-5 | C₈H₇NO₄S |
| 8 | dimethylguanidinovaleric acid | 107347-90-0 | C₈H₁₅N₃O₃ |
| | (DMGV) | | |
| 9 | N-acetyl-cadaverine | 32343-73-0 | C₇H₁₆N₂O |
| 10 | N-formylmethionine | 4289-98-9 | C₆H₁₁NO₃S |
| 11 | nicotinamide | 98-92-0 | C₆H₆N₂O |
| 12 | nicotinamide N-oxide | 1986-81-8 | C₆H₆N₂O₂ |
| 13 | N-methyl-GABA | 1119-48-8 | C₅H₁₁NO₂ |
| 14 | p-cresol glucuronide | 17680-99-8 | C₁₃H₁₆O₇ |
| 15 | p-cresol sulfate | 3233-58-7 | C₇H₈O₄S |
| 16 | phenylacetylalanine | 17966-65-3 | C₁₁H₁₃NO₃ |
| 17 | phenylacetylglutamate | — | C₁₃H₁₅NO₅ |
| | | | (structural formula |
| | | | shown in FIG. 5) |
| 18 | phenylacetylglutamine | 28047-15-6 | C₁₃H₁₆N₂O₄ |
| 19 | phenylacetylhistidine | — | C₆H₉N₃O₂C₈H₆O |
| | | | (structural formula |
| | | | shown in FIG. 6) |
| 20 | phenylacetylmethionine | — | C₅H₁₁NO₂SC₈H₆O |
| | | | (structural formula |
| | | | shown in FIG. 7) |
| 21 | phenylacetylserine | 65445-69-4 | C₁₁H₁₃NO₄ |
| 22 | phenylacetyltaurine | 33953-90-1 | C₁₀H₁₃NO₄S |
| 23 | phenylacetylthreonine | — | C₄H₉NO₃C₈H₆O |
| | | | (structural formula |
| | | | shown in FIG. 8) |
| 24 | trimethylamine N-oxide | 1184-78-7 | C₃H₉NO |
| 25 | xanthine | 69-89-6 | C₅H₄N₄O₂ |
| 26 | trizma acetate | 6850-28-8 | C₆H₁₅NO₅ |

### Example 2 Prediction Model for Colorectal Cancer

In the example, single biomarkers or a combination of multiple biomarkers screened in example 1 were used to establish prediction or diagnosis models for colorectal cancer. These models were used to distinguish colorectal cancer from non-colorectal cancer, to screen a patient with colorectal cancer from the population, or to predict whether an individual is a patient with colorectal cancer or the possibility of an individual suffering from colorectal cancer. Specific models were as follows.

1. Single Biomarkers

An R language software was used to process data. According to the grouping of patients with colorectal cancer and a non-colorectal cancer population, the concentration changes of 26 biomarkers in the urine samples of the patients with colorectal cancer and the non-colorectal cancer population were determined. All the detection results were subjected to an LASSO regression analysis to establish a mathematical model to predict whether an individual suffers from colorectal cancer, and the effectiveness of the regression model was evaluated by using a calibration curve and an ROC curve.

The analysis results showed that 26 biomarkers were significantly correlated with colorectal cancer. The analysis results were shown in Table 2 and Table 3.

TABLE 2
Comparison of correlation detection results of 26 biomarkers and colorectal cancer

| | | | | 95% CI | |
|---|---|---|---|---|---|
| Indexes | β | OR | p-value | Lower | Upper |
| 2-piperidinone | −5.302796177 | 0.004977656 | 0.0021997025 | −2.439395904 | −0.554916096 |
| 3-hydroxyanthranilate | #N/A | #N/A | 0.000195065 | −1.607767298 | −0.526480702 |
| 3-indoxyl sulfate | #N/A | #N/A | 0.123231485 | −1.158547932 | 0.140887932 |
| 4-hydroxyphenylacetyl- | 0.037986131 | 1.038716827 | 0.036132216 | −3.772580625 | −0.128923375 |
| glutamine | | | | | |
| 4-hydroxyphenylpyruvate | #N/A | #N/A | 0.036132216 | −3.772580625 | −0.128923375 |
| 5-hydroxyindole | #N/A | #N/A | 0.19451781 | −1.28588006 | 0.26590806 |
| glucuronide | | | | | |
| 6-hydroxyindole | #N/A | #N/A | 0.214792551 | −1.294562772 | 0.294750772 |
| sulfate | | | | | |
| dimethylguanidino | 0.481847118 | 1.619062241 | 0.002751023 | −3.214983555 | −0.704188445 |
| valeric acid | | | | | |
| N-acetyl-cadaverine | #N/A | #N/A | 0.006027911 | −5.694234145 | −1.003437854 |
| N-formylmethionine | #N/A | #N/A | 0.006200771 | −1.193531098 | −0.204336902 |
| nicotinamide | −1.525090436 | 0.217601377 | 0.011443056 | 0.132060748 | 1.012671252 |
| nicotinamide N-oxide | #N/A | #N/A | 0.0000369156 | 0.543634687 | 1.434193313 |
| N-methyl-4- | −1.007770314 | 0.365031979 | 0.000151406 | 0.380868329 | 1.147055671 |
| aminobutyric acid | | | | | |
| p-cresol glucuronide | −0.035366893 | 0.965251207 | 0.005961446 | −10.71959689 | −1.875015108 |
| p-cresol sulfate | −0.021798367 | 0.978437501 | 0.004011742 | −3.683079137 | −0.724172863 |
| phenylacetylalanine | −0.190202421 | 0.826791757 | 0.021098845 | −3.439994011 | −0.286757989 |
| phenylacetylglutamate | #N/A | #N/A | 0.027185752 | −2.7677993445 | −0.170452655 |
| phenylacetylglutamine | 0.858050782 | 2.358558865 | 1.02818E−05 | −1.93344453 | −0.78233147 |
| phenylacetylhistidine | #N/A | #N/A | 0.0015908 | −2.25387961 | −0.54944039 |
| phenylacetylmethionine | −0.118805316 | 0.88798066 | 0.001919024 | −3.178504783 | −0.750631217 |
| phenylacetylserine | #N/A | #N/A | 0.00005738 | −2.447360211 | −0.890135789 |
| phenylacetyltaurine | #N/A | #N/A | 0.017478353 | −2.948912433 | −0.294979567 |
| phenylacetylthreonine | 0.597275285 | 1.817160804 | 0.002659366 | −2.782042717 | −0.609085283 |
| trimethylamine N-oxide | #N/A | #N/A | 0.00416445 | −0.660183306 | −0.127504694 |
| xanthine | #N/A | #N/A | 0.828967916 | −0.657362597 | 0.818398597 |
| trizma acetate | #N/A | #N/A | 0.000041591 | −111.298499 | −42.38852896 |

TABLE 3
ROC analysis results of single biomarkers

| Serial | | AUC | | | Cut- |
|---|---|---|---|---|---|
| No. | Biomarkers | value | Sensitivity | Specificity | off |
| 1 | 2-piperidinone | 0.7156 | 0.925 | 0.68 | 0.72 |
| 2 | 3-hydroxyanthranilate | 0.7218 | 0.53175 | 0.52 | 0.82 |
| 3 | 3-indoxyl sulfate | 0.7096 | 0.8711 | 0.62 | 0.76 |
| 4 | 4-hydroxy- | 0.7036 | 1.24985 | 0.74 | 0.62 |
| | phenylacetylglutamine | | | | |
| 5 | 4-hydroxyphenylpyruvate | 0.7668 | 0.97835 | 0.72 | 0.76 |
| 6 | 5-hydroxyindole | 0.7112 | 0.3662 | 0.46 | 0.96 |
| | glucuronide | | | | |
| 7 | 6-hydroxyindole sulfate | 0.6864 | 0.63085 | 0.48 | 0.86 |
| 8 | dimethylguanidinovaleric | 0.722 | 0.2471 | 0.58 | 0.82 |
| | acid | | | | |
| 9 | N-acetyl-cadaverine | 0.7796 | 0.582 | 0.54 | 0.9 |
| 10 | N-formylmethionine | 0.6568 | 0.20645 | 0.28 | 0.98 |
| 11 | nicotinamide | 0.6324 | 2.27625 | 0.32 | 0.98 |
| 12 | nicotinamide N-oxide | 0.772 | 0.1686 | 0.88 | 0.58 |
| 13 | N-methyl-4-aminobutyric | 0.7444 | 1.1929 | 0.62 | 0.78 |
| | acid | | | | |
| 14 | p-cresol glucuronide | 0.7836 | 0.86 | 0.64 | 0.64 |
| 15 | p-cresol sulfate | 0.7348 | 0.7536 | 0.64 | 0.82 |
| 16 | phenylacetylalanine | 0.7428 | 1.6654 | 0.8 | 0.58 |
| 17 | phenylacetylglutamate | 0.6988 | 1.0442 | 0.68 | 0.72 |
| 18 | phenylacetylglutamine | 0.7876 | 0.5643 | 0.62 | 0.84 |
| 19 | phenylacetylhistidine | 0.7478 | 0.96145 | 0.72 | 0.7 |
| 20 | phenylacetylmethionine | 0.7768 | 0.73925 | 0.7 | 0.78 |
| 21 | phenylacetylserine | 0.78 | 1.116 | 0.74 | 0.68 |
| 22 | phenylacetyltaurine | 0.6968 | 0.6231 | 0.5 | 0.84 |
| 23 | phenylacetylthreonine | 0.7352 | 1.21925 | 0.72 | 0.7 |
| 24 | trimethylamine N-oxide | 0.6708 | 0.9524 | 0.66 | 0.7 |
| 25 | xanthine | 0.774 | 0.8734 | 0.78 | 0.68 |
| 26 | trizma acetate | 0.7354 | 0.72 | 0.86 | 0.72 |

The correlation between the concentration changes of the 26 biomarkers and the colorectal cancer can be distinguished by OR values, p-values and the like in Table 2, and also can be distinguished by AUC values and the like in Table 3, wherein the OR values and the AUC values were most visual and obvious. The higher OR value indicated that the patients with colorectal cancer had a greater impact on the index compared with non-colorectal cancer patients, and the index exposure was more obvious. The higher AUC value indicated that the biomarker could more accurately distinguish between the colorectal cancer population and the non-colorectal cancer population.

It can be seen from Table 2 that the concentration changes of the 26 biomarkers were obviously correlated with colorectal cancer, wherein the phenylacetylglutamine had the highest correlation, with an OR value of 2.36, followed by phenylacetylthreonine, with an OR value of 1.82.

It can be seen from Table 3 that the AUC value of the concentration change of any of 26 biomarkers used alone to distinguish the colorectal cancer population and the non-colorectal cancer population can reach 0.63 or more, with high accuracy. The phenylacetylglutamine had the highest AUC value of 0.7876, followed by p-cresol glucuronide having the AUC value of 0.7836.

2. Combination of Multiple Biomarkers

Although a single biomarker can also be used to distinguish urine samples of colorectal cancer from non-colorectal cancer or predict colorectal cancer. It is generally more accurate to combine multiple biomarkers for distinguishment or prediction.

However, the single biomarker with higher accuracy in predicting colorectal cancer does not necessarily play a larger role in the combination when combined with other one or more biomarkers. At the same time, the more number of the biomarkers does not indicate higher accuracy of prediction (AUC value) of the combination. Therefore, a large number of verification experiments are required.

Since the AUC and OR values of the biomarkers are biased toward evaluating the relative importance of the variables in the statistical models and are not suitable for constructing a model for the preferred variables, the example preferably used 2, 3, 5, 10, 20, and 26 biomarkers with the highest concentration fold change in the urine samples of colorectal cancer and non-colorectal cancer to construct a diagnostic model for colorectal cancer. The concentration fold change (fold change=expression mean value of disease sample divided by expression mean value of normal sample) of the 26 biomarkers in the urine samples of colorectal cancer and non-colorectal cancer ranked from high to low, and the results were shown in Table 4.

TABLE 4
Ranking of concentration fold changes of 26 biomarkers in urine
samples of colorectal cancer and non-colorectal cancer

| | | Fold | | |
|---|---|---|---|---|
| Rank | Biomarkers | Change | T-tests | AUC |
| 1 | p-cresol sulfate | 5.0115 | 4.1426E−11 | 0.7348 |
| 2 | phenylacetylthreonine | 4.8447 | 6.0512E−10 | 0.7352 |
| 3 | N-methyl-4-aminobutyric | 3.0586 | 7.534E−7 | 0.7444 |
| | acid | | | |
| 4 | 4-hydroxyphenylpyruvate | 2.8178 | 2.2337E−5 | 0.7668 |
| 5 | phenylacetylmethionine | 2.7238 | 3.8753E−7 | 0.7768 |
| 6 | p-cresol glucuronide | 2.7028 | 1.7998E−7 | 0.7836 |
| 7 | nicotinamide | 2.0965 | 1.9958E−6 | 0.6324 |
| 8 | phenylacetylalanine | 2.0369 | 7.5589E−6 | 0.7428 |
| 9 | phenylacetylglutamine | 1.8305 | 4.9111E−8 | 0.7876 |
| 10 | dimethylguanidinovaleric | 1.8246 | 7.6888E−5 | 0.722 |
| | acid | | | |
| 11 | 3-hydroxyanthranilate | 1.7392 | 8.2523E−4 | 0.7218 |
| 12 | 5-hydroxyindole | 1.643 | 3.063E−4 | 0.7112 |
| | glucuronide | | | |
| 13 | phenylacetylglutamate | 1.6132 | 1.085E−7 | 0.6988 |
| 14 | phenylacetylhistidine | 1.5252 | 3.3237E−5 | 0.7478 |
| 15 | 2-piperidinone | 1.4667 | 1.1365E−4 | 0.7156 |
| 16 | N-formylmethionine | 1.3568 | 4.7596E−4 | 0.6568 |
| 17 | phenylacetyltaurine | 1.2161 | 1.9028E−5 | 0.6968 |
| 18 | 3-indoxyl sulfate | 0.98732 | 3.4274E−4 | 0.7096 |
| 19 | 6-hydroxyindole sulfate | 0.92086 | 0.0019052 | 0.6864 |
| 20 | trimethylamine N-oxide | 0.77014 | 7.5794E−5 | 0.6708 |
| 21 | 4-hydroxyphenyl- | −0.5916 | 0.34593 | 0.7036 |
| | acetylglutamine | | | |
| 22 | N-acetyl-cadaverine | −0.77292 | 0.18073 | 0.7796 |
| 23 | trizma acetate | −0.83338 | 0.0016428 | 0.7354 |
| 24 | xanthine | −1.0127 | 2.1818E−5 | 0.774 |
| 25 | nicotinamide N-oxide | −1.2215 | 0.003826 | 0.772 |
| 26 | phenylacetylserine | −1.7863 | 0.001003 | 0.78 |

According to the concentration fold changes of the 26 biomarkers in the urine samples of colorectal cancer and non-colorectal cancer provided in Table 4, 2, 3, 5, 10, 20, and 26 biomarkers of the 26 biomarkers were selected respectively in the example to construct a diagnostic model of colorectal cancer through random forest.

The 2 biomarkers were the first and second biomarkers (p-cresol sulfate and phenylacetylthreonine) in Table 4. In the constructed random forest model, the information gain ratio (GINI coefficient) of the p-cresol sulfate was 25.31 and the mean decrease accuracy was 21.17; and the GINI coefficient of the phenylacetylthreonine was 24.22 and the mean decrease accuracy was 16.71.

The 3 biomarkers were the first to third biomarkers in Table 4. In the constructed random forest model, the GINI coefficient of the p-cresol sulfate was 15.43 and the mean decrease accuracy was 16.37; the GINI coefficient of the phenylacetylthreonine was 15.75 and the mean decrease accuracy was 15.04; and the GINI coefficient of the N-methyl-4-aminobutyric acid was 18.33 and the mean decrease accuracy was 24.42.

The 5 biomarkers were the first to fifth biomarkers in Table 4. In the constructed random forest model, the GINI coefficient of the p-cresol sulfate was 7.86 and the mean decrease accuracy was 10.99; the GINI coefficient of the phenylacetylthreonine was 6.39 and the mean decrease accuracy was 5.58; the GINI coefficient of the N-methyl-4-aminobutyric acid was 13.73 and the mean decrease accuracy was 25.36; the GINI coefficient of the 4-hydroxyphenylpyruvate was 10.43 and the mean decrease accuracy was 45.38; and the GINI coefficient of the phenylacetylmethionine was 11.05 and the mean decrease accuracy was 18.74.

The 10 biomarkers were the first to tenth biomarkers in Table 4. In the constructed random forest model, the GINI coefficient of the p-cresol sulfate was 3.64 and the mean decrease accuracy was 7.56; the GINI coefficient of the phenylacetylthreonine was 2.46 and the mean decrease accuracy was 4.80; the GINI coefficient of the N-methyl-4-aminobutyric acid was 8.04 and the mean decrease accuracy was 18.60; the GINI coefficient of the 4-hydroxyphenylpyruvate was 6.25 and the mean decrease accuracy was 12.60; the GINI coefficient of the phenylacetylmethionine was 6.26 and the mean decrease accuracy was 12.85; the GINI coefficient of the p-cresol glucuronide was 5.20 and the mean decrease accuracy was 11.07; the GINI coefficient of the nicotinamide was 6.56 and the mean decrease accuracy was 12.51; the GINI coefficient of the phenylacetylalanine was 3.18 and the mean decrease accuracy was 6.30; the GINI coefficient of the phenylacetylglutamine was 4.47 and the mean decrease accuracy was 6.83; and the GINI coefficient of the dimethylguanidinovaleric acid was 3.43 and the mean decrease accuracy was 9.16.

The 20 biomarkers were the first to twentieth biomarkers in Table 4. In the constructed random forest model, the GINI coefficient of the p-cresol sulfate was 2.36 and the mean decrease accuracy was 6.21; the GINI coefficient of the phenylacetylthreonine was 1.73 and the mean decrease accuracy was 4.02; the GINI coefficient of the N-methyl-4-aminobutyric acid was 5.92 and the mean decrease accuracy was 16.23; the GINI coefficient of the 4-hydroxyphenylpyruvate was 4.10 and the mean decrease accuracy was 9.28; the GINI coefficient of the phenylacetylmethionine was 3.79 and the mean decrease accuracy was 10.13; the GINI coefficient of the p-cresol glucuronide was 3.77 and the mean decrease accuracy was 9.49; the GINI coefficient of the nicotinamide was 4.67 and the mean decrease accuracy was 11.61; the GINI coefficient of the phenylacetylalanine was 2.26 and the mean decrease accuracy was 5.84; the GINI coefficient of the phenylacetylglutamine was 2.67 and the mean decrease accuracy was 7.71; the GINI coefficient of the dimethylguanidinovaleric acid was 2.00 and the mean decrease accuracy was 7.77; the GINI coefficient of the 3-hydroxyanthranilate was 2.03 and the mean decrease accuracy was 4.32; the GINI coefficient of the 5-hydroxyindole glucuronide was 2.69 and the mean decrease accuracy was 5.66; the GINI coefficient of the phenylacetylglutamate was 1.59 and the mean decrease accuracy was 4.38; the GINI coefficient of the phenylacetylhistidine was 1.62 and the mean decrease accuracy was 4.96; the GINI coefficient of the 2-piperidinone was 1.57 and the mean decrease accuracy was 1.85; the GINI coefficient of the N-formylmethionine was 1.45 and the mean decrease accuracy was 2.81; the GINI coefficient of the phenylacetyltaurine was 1.28 and the mean decrease accuracy was 0.79; the GINI coefficient of the 3-indoxyl sulfate was 1.41 and the mean decrease accuracy was 3.51; the GINI coefficient of the 6-hydroxyindole sulfate was 1.57 and the mean decrease accuracy was 1.93; and the GINI coefficient of the trimethylamine N-oxide was 1.02 and the mean decrease accuracy was 2.61.

The 26 biomarkers were the first to twenty-sixth biomarkers in Table 4. In the constructed random forest model, the GINI coefficient of the p-cresol sulfate was 1.69 and the mean decrease accuracy was 7.04; the GINI coefficient of the phenylacetylthreonine was 1.04 and the mean decrease accuracy was 2.80; the GINI coefficient of the N-methyl-4-aminobutyric acid was 3.57 and the mean decrease accuracy was 12.93; the GINI coefficient of the 4-hydroxyphenylpyruvate was 2.45 and the mean decrease accuracy was 5.50; the GINI coefficient of the phenylacetylmethionine was 2.68 and the mean decrease accuracy was 7.68; the GINI coefficient of the p-cresol glucuronide was 2.61 and the mean decrease accuracy was 8.31; the GINI coefficient of the nicotinamide was 2.56 and the mean decrease accuracy was 8.02; the GINI coefficient of the phenylacetylalanine was 1.47 and the mean decrease accuracy was 4.84; the GINI coefficient of the phenylacetylglutamine was 1.83 and the mean decrease accuracy was 5.74; the GINI coefficient of the dimethylguanidinovaleric acid was 1.34 and the mean decrease accuracy was 3.76; the GINI coefficient of the 3-hydroxyanthranilate was 1.14 and the mean decrease accuracy was 4.11; the GINI coefficient of the 5-hydroxyindole glucuronide was 1.76 and the mean decrease accuracy was 4.39; the GINI coefficient of the phenylacetylglutamate was 0.88 and the mean decrease accuracy was 3.11; the GINI coefficient of the phenylacetylhistidine was 1.00 and the mean decrease accuracy was 4.79; the GINI coefficient of the 2-piperidinone was 1.20 and the mean decrease accuracy was 1.80; the GINI coefficient of the N-formylmethionine was 0.79 and the mean decrease accuracy was 2.15; the GINI coefficient of the phenylacetyltaurine was 0.58 and the mean decrease accuracy was 2.70; the GINI coefficient of the 3-indoxyl sulfate was 0.96 and the mean decrease accuracy was 3.64; the GINI coefficient of the 6-hydroxyindole sulfate was 0.73 and the mean decrease accuracy was 2.70; the GINI coefficient of the trimethylamine N-oxide was 0.74 and the mean decrease accuracy was 2.33; the GINI coefficient of the 4-hydroxyphenylacetylglutamine was 0.83 and the mean decrease accuracy was 4.61; the GINI coefficient of the N-acetyl-cadaverine was 2.22 and the mean decrease accuracy was 7.72; the GINI coefficient of the trizma acetate was 2.48 and the mean decrease accuracy was 8.06; the GINI coefficient of the xanthine was 2.70 and the mean decrease accuracy was 8.67; the GINI coefficient of the nicotinamide N-oxide was 8.21 and the mean decrease accuracy was 16.94; and the GINI coefficient of the phenylacetylserine was 2.01 and the mean decrease accuracy was 7.16.

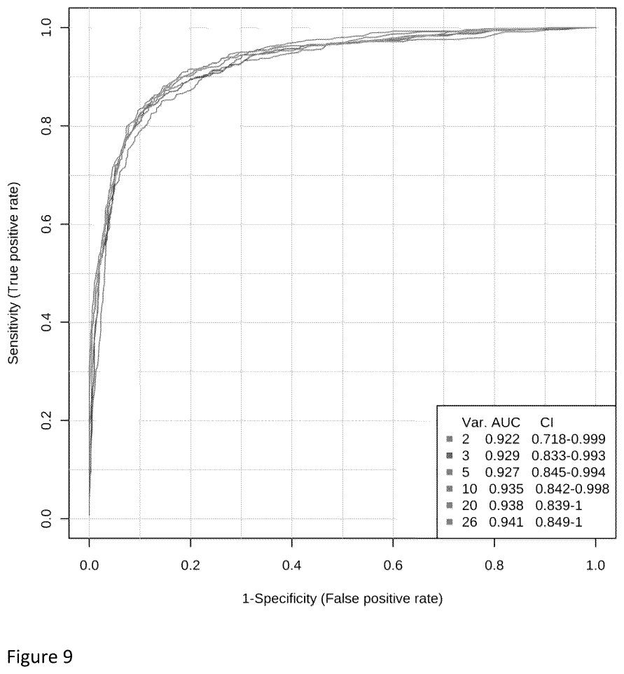

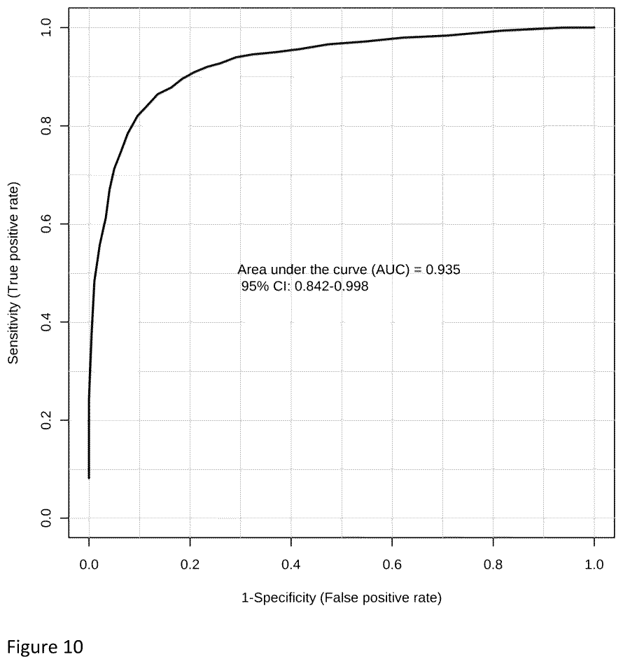

The AUC value and 95% confidence interval (CI) of the six random forest diagnostic models constructed with the above 2, 3, 5, 10, 20, and 26 biomarkers were calculated respectively, and the results were shown in FIG. 9.

It can be seen from FIG. 9 that the AUC value of the model constructed by selecting two biomarkers with the highest ranking among the 26 biomarkers can only reach 0.922, and the 95% CI was 0.718-0.999. As the number of the selected biomarkers increased, the AUC value gradually increased, and the 95% CI gradually decreased. When 10 biomarkers were selected to construct a diagnostic model for colorectal cancer, the AUC value reached 0.935 and the 95% CI was 0.842-0.998. However, when the number of the biomarkers further rose to 20 or 26, the space for AUC to continue to rise was very limited, and the confidence interval became larger. In addition, compared with 20 and 26 biomarkers, the use of 10 biomarkers to construct a model can reduce the number of variables and reduce the complexity of the model. Therefore, it is preferred to use the top 10 biomarkers in Table 4 to construct the diagnostic model for colorectal cancer, and thus very good prediction accuracy can be achieved and the model is simpler and more convenient.

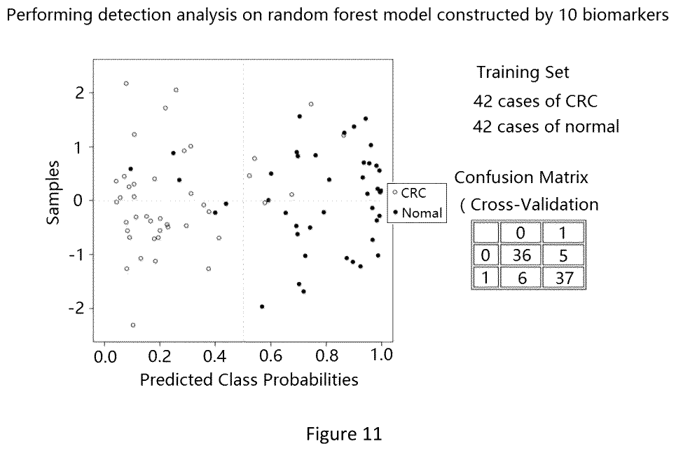

42 clinically known patients with colorectal cancer and 42 non-colorectal cancer patients were taken as the total data set to detect the biomarker detection values of the urine samples. The analysis was performed through the random forest model of 10 biomarkers. The analysis map was shown in FIG. 11. It can be seen from FIG. 11 that when the random forest model constructed with the 10 biomarkers was used to predict colorectal cancer, there would be some errors (of course, the errors were unavoidable). Among the 42 patients with colorectal cancer, 37 cases were detected. Among 42 non-colorectal cancer patients, 5 cases were classified as patients with colorectal cancer. The accuracy rate was 88%. It can be seen from FIG. 11, when a predictive value p was greater than 0.5, an individual was predicted to have a high probability of colorectal cancer; and when a predictive value p was less than 0.5, an individual was predicted to have a low probability of colorectal cancer.

The 10 biomarkers of the top 10 biomarkers of fold change were used for multivariate regression analysis to establish a logistic regression evaluation model to predict whether an individual suffered from colorectal cancer:

Z=4-hydroxyphenylpyruvate*0.037986+dimethylguanidinovaleric acid*0.4818−N-methyl-4-aminobutyric acid*1.0077−nicotinamide*1.525−p-cresol glucuronide*0.0353−p-cresol sulfate*0.021798−phenylacetylalanine*0.1902+phenylacetylglutamine*0.858−phenylacetylmethionine*0.118805+phenylacetylthreonine*0.59727+0.7486,

$$p = \frac{1}{1 + {e}^{z}}$$

wherein e is the base of the natural logarithm; and p is a predictive value for predicting whether an individual suffers from colorectal cancer and the name of the biomarker represents the relative abundance of the corresponding biomarker in a urine sample, that is, a peak area of the biomarker in a detection spectrum obtained by an ultra-performance liquid chromatography-tandem mass spectrometry.

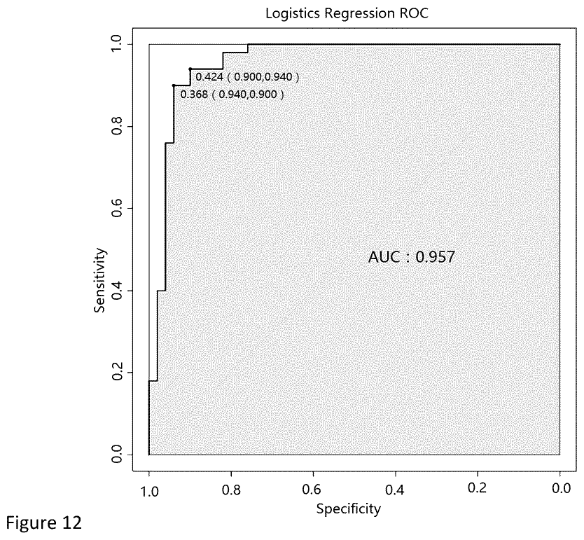

The ROC curve of the logistic regression model to predict whether an individual suffers from colorectal cancer provided in the example was shown in FIG. 12. The AUC value reached 0.957 and was significantly higher than that of the random forest model of 10 biomarkers.

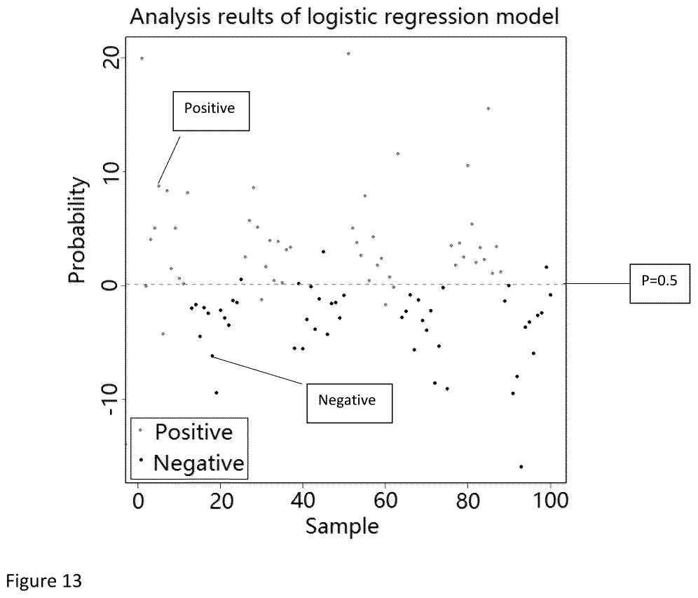

The logistic regression model was used to predict whether an individual suffered from colorectal cancer. 50 clinically known patients with colorectal cancer and 50 non-colorectal cancer patients were taken as the total data set for analysis. The analysis results were shown in FIG. 13 and Table 5.

TABLE 5
Analysis results of model for predicting whether individual
suffering from colorectal cancer
Analysis results of logistic regression model

| Actual prediction | Negative | Positive |
|---|---|---|
| Negative | 46 | 4 |
| Positive | 5 | 45 |

It can be seen from FIG. 13 and Table 5 that the logistic regression evaluation model constructed by the 10 biomarkers to predict whether an individual suffered from colorectal cancer was used for analysis. Among 50 patients with colorectal cancer, 45 were detected. Among 50 non-colorectal cancer patients, 5 cases were classified as patients with colorectal cancer. The accuracy rate reached 90% or more, and thus was improved.

It can be seen from FIG. 13, p of 0.5 can be used as a dividing point for determination. When a predictive value p was greater than 0.5, an individual was predicted to have a high probability of colorectal cancer; and when a predictive value p was less than 0.5, an individual was predicted to have a low probability of colorectal cancer.

### Example 3 Evaluation of Model for Predicting Colorectal Cancer

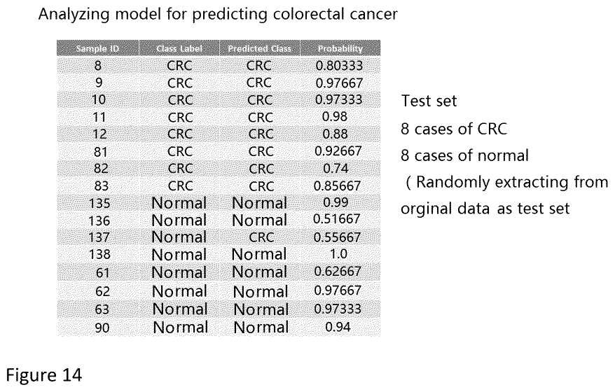

In the example, the accuracy of clinical application of the model for predicting colorectal cancer constructed in example 2 was evaluated. The above 42 patients with colorectal cancer and 42 non-colorectal cancer patients were taken as the total data set, from which 8 patients with CRC and 8 normal people (non-CRC patients) were randomly selected, and urine samples were taken. The relative abundance of the 10 biomarkers in the model was measured according to the sample processing method in example 1, so as to calculate the predictive value p through the model and predict whether an individual suffers from colorectal cancer. The results were shown in FIG. 14.

It can be seen from FIG. 14 that all the 8 patients with colorectal cancer were detected, and one of the 8 normal people was predicted to suffer from colorectal cancer, with an accuracy rate of 93.75%.

All the patents and publications mentioned in the description of the present disclosure indicate that these are public technologies in the art and can be used by the present disclosure. All the patents and publications cited herein are listed in the references, just as each publication is specifically referenced separately. The present disclosure described herein can be realized in the absence of any one element or multiple elements, one restriction or multiple restrictions, where the limitation is not specifically described here. For example, in each example, the terms “comprise”, “substantially composed of” and “composed of” can be replaced by the remaining two terms of either. The so-called “a” here only means “a kind”, not excluding only one, but also can indicate two or more. The terms and expressions used herein are descriptive, without limitation. Besides, there is no intention to indicate that these terms and interpretations described in the description exclude any equivalent features. However, it can be known that any appropriate changes or modifications can be made within the scope of the present disclosure and claims. It can be understood that the examples described in the present disclosure are some preferred examples and features. A person skilled in the art can make some modifications and changes according to the essence of the description of the present disclosure. These modifications and changes are also considered to fall within the scope of the present disclosure and the scope limited by independent claims and dependent claims.

1-20. (canceled)

21. A system for predicting whether an individual suffers from colorectal cancer, wherein the system comprises a data analysis module; and the data analysis module is configured to analyze a detection value of a biomarker, and the biomarker consists of 4-hydroxyphenylpyruvate, dimethylguanidinovaleric acid, N-methyl-4-aminobutyric acid, nicotinamide, p-cresol glucuronide, p-cresol sulfate, phenylacetylalanine, phenylacetylglutamine, phenylacetylmethionine, and phenylacetylthreonine.

22. The system according to claim 21, wherein the detection value of the biomarker is obtained by detecting the biomarker in a urine sample.

23. The system according to claim 22, wherein the detection value of the biomarker is obtained by detecting the presence or relative abundance or concentration of the biomarker in the urine sample of the individual.

24. The system according to claim 23, wherein the data analysis module adopts a random forest or a logistic regression equation to construct a model for analysis.

25. The system according to claim 24, wherein the data analysis module calculates a predictive value for predicting whether an individual suffers from colorectal cancer by substituting the detection value of the biomarker into the logistic regression equation to evaluate whether the individual suffers from the colorectal cancer.

26. The system according to claim 25, wherein the logistic regression equation is:
Z=4-hydroxyphenylpyruvate*0.037986+dimethylguanidinovaleric acid*0.4818−N-methyl-4-aminobutyric acid*1.0077−nicotinamide*1.525−p-cresol glucuronide*0.0353−p-cresol sulfate*0.021798−phenylacetylalanine*0.1902+phenylacetylglutamine*0.858−phenylacetylmethionine*0.118805+phenylacetylthreonine*0.59727+0.7486,
$$p = \frac{1}{1 + {e}^{z}}$$
wherein e is the base of the natural logarithm; and p is the predictive value for predicting whether the individual suffers from the colorectal cancer.

27. The system according to claim 26, wherein when p is greater than 0.5, the individual is predicted to have a high probability of colorectal cancer; and when p is less than 0.5, the individual is predicted to have a low probability of colorectal cancer.
